(19) United States
(12) Patent Application Publication
(10) Pub. No.: US 2019/0076668 A1
(43) Pub. Date: Mar. 14, 2019

(54) MEDICAL RADIATION DEVICE

(71) Applicant: IGC MEDICAL LLC, Laurel, MD (US)

(72) Inventors: Mary Elizabeth McLay-Brown, Daytona Beach, FL (US); Mikhail A. Maiorov, Titusville, NJ (US)

(73) Assignee: IGC MEDICAL LLC, LAUREL, MD (US)

(21) Appl. No.: 16/055,051

(22) Filed: Aug. 4, 2018

Related U.S. Application Data
(60) Provisional application No. 62/605,308, filed on Aug. 8, 2017.

(51) Int. Cl. A61N 5/06

(52) U.S. Cl. CPC A61N 5/06; A61N 2005/067

(57) ABSTRACT

An improved low power mid-infrared diode laser device and method for non-invasive treatment of biological tissue and internal organs. The improved digital over analog system includes a treatment probe with extended cone containing an integrated pointing laser and power monitor. The laser is contained in the base unit and is delivered to the probe through armored fiber optic cable and is pulsed at 7.83 Hz. The improved design monitors a portion of the beam which is deflected by a dichroic mirror creating a reference signal measuring a fraction of the power at the exit of the module for real time power adjustment of the laser driver, detection of diode failure, and self-calibration of the device. A diffuser is utilized to create an output beam required for precise therapeutic application spot size and intensity distribution of the laser energy.

### FIELD OF INVENTION

The New Device has medical applications in methods of treating inflammation in a human or animal subject. The new device offers treatment of inflammation associated with trauma and disease without invasive procedures and demonstrates a non-invasive and drug free treatment. There are no known medical modalities with these properties.

### BACKGROUND

Therapeutic lasers are well known in the art. Light is able to penetrate tissue and deposit energy thru the absorptive properties of the tissue irradiated. Historically these lasers treat a variety of disorders related to localized areas of the body such as arthritic joint pain; carpal tunnel syndrome; pain from adipose tissue removal; pain following surgery and other localized afflictions. These lasers operate at different wavelengths, mostly in the visible end of the electromagnetic spectrum from approximately 600 nm to 740/770 nm and the invisible part of the spectrum from approximately 770 nm to 1000 nm. A few operate in the area of approximately 1064 nm specifically for dermatology applications in the removal of wrinkles and 1917 nm specifically for immune system stimulation. These inventions offer therapeutic value in treating the symptom(s) of the disease, trauma, or infirmity. The new Device operates at areas contained within a bandwidth from approximately 1900 nm thru approximately 3200 nm, specifically targeting certain cells, giving the new device the capability of expanding its operation to treat beyond the devices that have been invented into treating inflammation associated with trauma and disease. It is important to note that the wavelengths employed in the new device are very different in their scientific properties than the rest of the visible and infrared radiation band and it is these differences which allow the new device to be novel in itself and in its medical applications.

### SUMMARY

The invention described herein demonstrates a novel digital radiation device that has therapeutic medical applications for the treatment of inflammation associated with trauma and disease with improvements over existing devices.

### BRIEF DESCRIPTION OF THE DRAWINGS

FIG. 1 shows the General schematic of the laser module operating at a functional wavelength; a laser chip 10, a Wavelength-stabilized element with collimating optics 12, an Optical beam combining functional laser with red pointer 14, and Beam splitter with reference signal 16 and the Output beam 18.

FIG. 2 shows a Schematic of the laser system 20 based on the laser module shown in FIG. 1, plus the connecting fiber optic cable 22 connecting the laser module 20 to the Hand piece or treatment probe 24 and the output beam 18.

FIG. 3 shows the modified hand piece 24 which provides improved power accuracy due to the Reference signal 26 which provides for power monitoring and power adjustment.

FIG. 4 shows the Evolution of the wavelength stabilization designs showing the AR coated facet 10 with collimating optics 28 and selective reflection element 30.

FIG. 5 shows the Advancement of the wavelength stabilizing design with the AR coated chip and reflective element as one piece 30, providing improved wavelength stabilization.

FIG. 5 shows even further advancement in the wavelength stabilizing element with the AR coated chip, the reflective element 30 and the Gain chip 32 connected as one piece to further improve the wavelength stabilizing element.

FIG. 6 shows the wavelength stabilizing element 30 attached to the collimating lens 28 and the gain chip 32 to be used as an integrated element to improve efficacy.

FIG. 7 shows collimated beam imperfections 36 versus dimensional errors in optical elements 38

FIG. 8 shows the laser with integrated optics (wavelength stabilization and collimation 38) with diffuser 40 used to minimize the effects of beam collimation quality on the resulting intensity distribution for use in therapeutic applications.

FIG. 9 shows the combining the features of the design in a hand piece or a handheld laser. This is a compact solution for an advanced hand piece/treatment probe or a complete handheld laser system using the wavelength stabilized laser 12, the red laser 14, the diffuser 40, and the dichroic mirror 42.

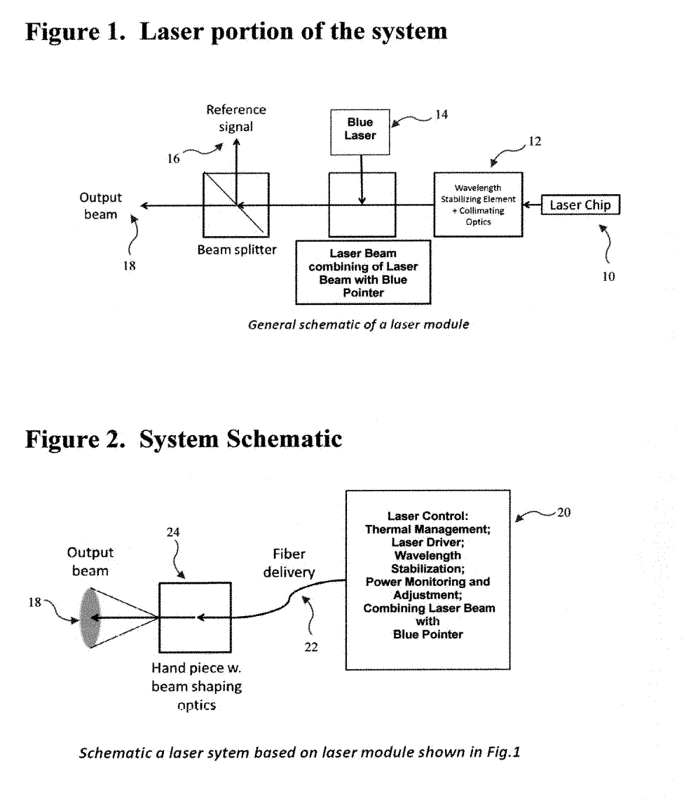

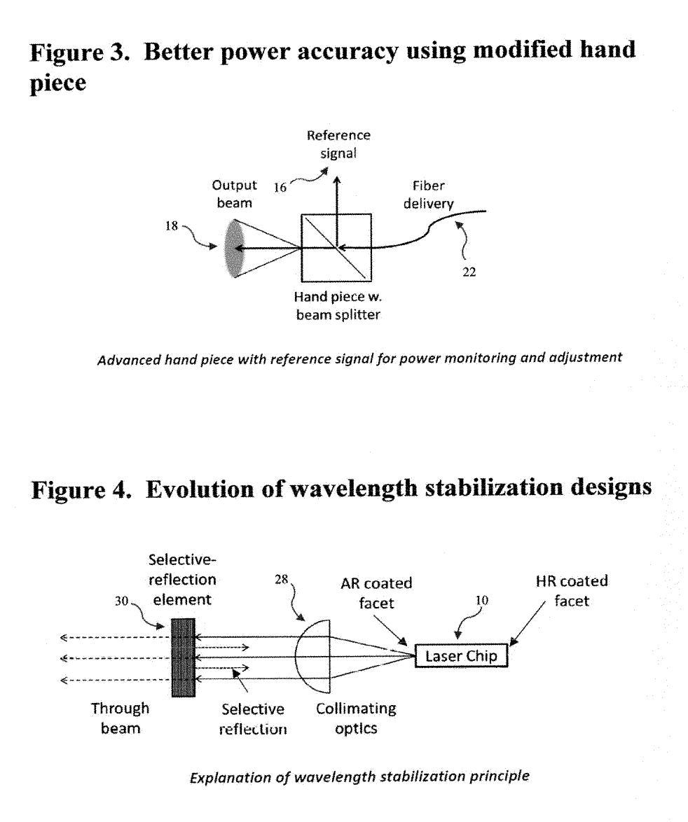

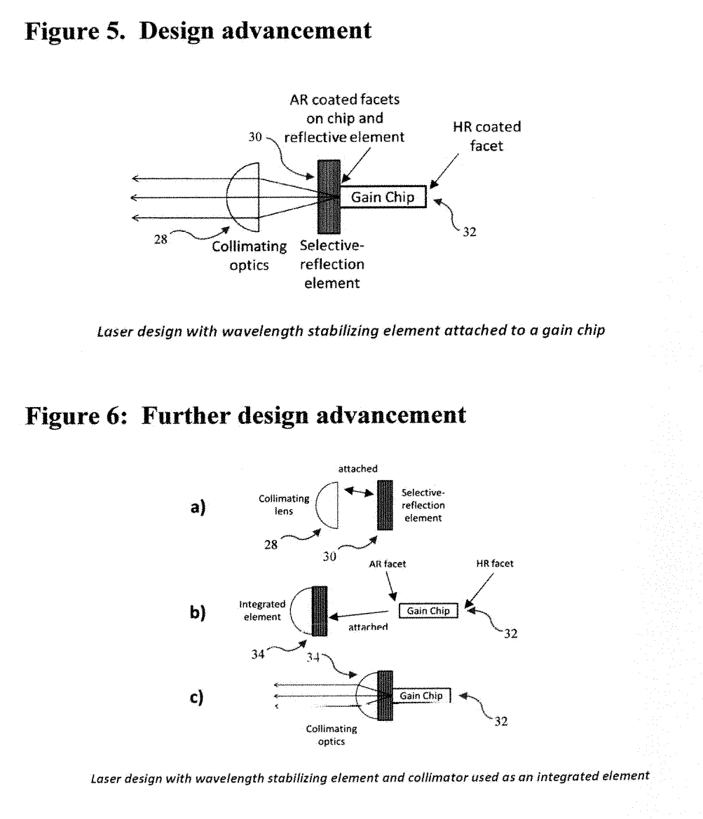

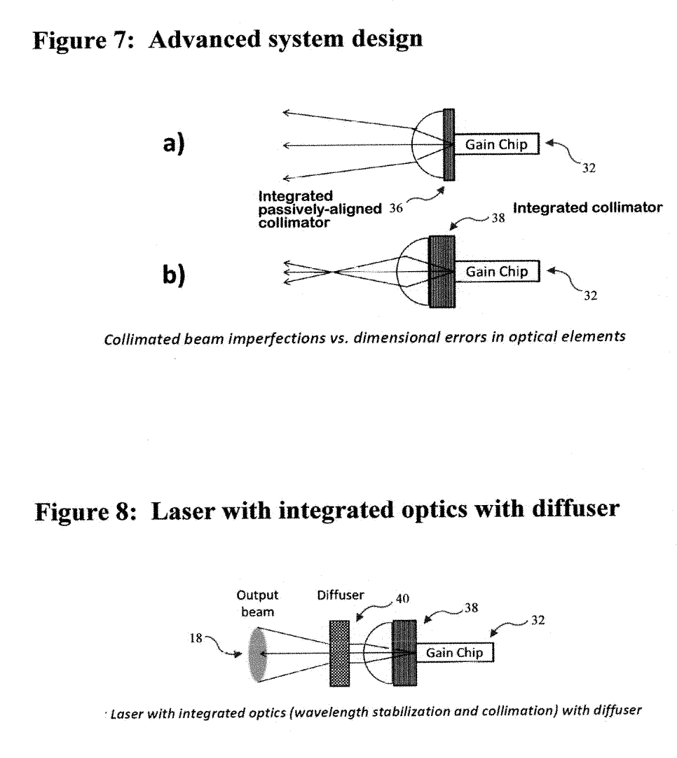

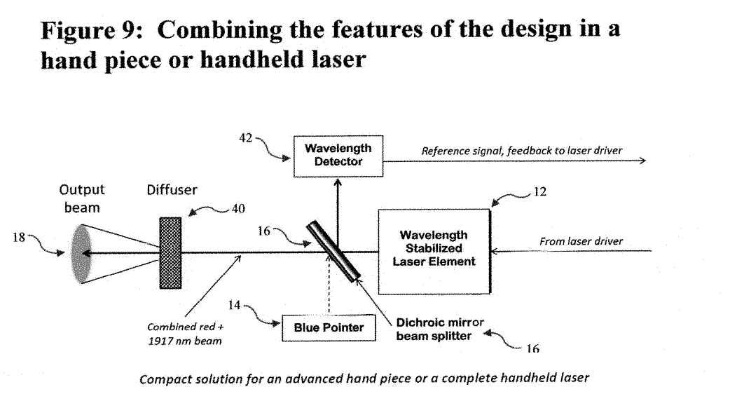

### DETAILED DESCRIPTION

The device generates and emits a non-focused beam of radiation in the infrared area of the electromagnetic spectrum with a bandwidth of approximately 1900 nm thru approximately 3200 nm with an adjustable pulse duration and pulse frequency, with 7.83 Hz frequency operation preferred. The preferred embodiment of the light source is an InP- or GaSb-based laser diode also known as an infrared laser diode or a semiconductor diode laser. Other sources of the laser include Light Emitting Diodes, LED's, and vertical cavity surface emitting laser, VCSEL. The invention is low power operating at less than 50 mW. The treatment method is non-invasive and topical in nature providing a localized and systemic treatment effect. The system design provides for a table top three-piece system consisting of the handheld treatment probe or hand piece, the fiber optic connecting cable, and the main system laser control center. Additional versions include a compact design combining the features of the design in the hand piece for better power accuracy or a handheld laser as a one-piece system. The new device is essentially maintenance free and allows for power adjustment in real time by measuring power in the deflected beam creating a reference signal which will adjust the laser driver output in real time, timely detection of chip failure, and device self-calibration. A photodiode is used for power measurement.

The present low power invention comprises a novel method of 1) controlling and locking the selected wavelength for delivery to the patient, 2) maintaining selected power levels while continuously monitoring chip/diode function and laser driver output in real-time, 3) continuously providing self-calibration of the device, 4) providing memory and data storage, 5) sending data via the internet, 6) unique pulse rate selection, and 7) therapeutic operation in the area of the electromagnetic spectrum where the radiation is absorbed via the water molecule instead of the areas where the water is transparent to the radiation. An advanced embodiment of the device will allow for the selected wavelength to be locked within +/−2 nm by using a wavelength stabilizing element in combination with collimated optics.

In preferred embodiments, the new device provides for a wavelength stabilized laser module operating at a selected wavelength with integrated pointing laser and power monitor measuring fraction of the power at the exit of the module to adjust the power, detect chip failure and provide device self-calibration in real time. The same features described above can be employed in a compact hand piece and also used as a handheld one-piece laser. The wavelength is adjustable in a wide wavelength range by using different laser diodes operating at different wavelengths, and by a wavelength stabilizing element, that narrows the emission spectrum of a laser diode, and fixes the wavelength at any desired value. The wavelength is a pre-set value—it cannot be changed for a particular device, but one can decide BEFORE building the device what the wavelength should be and select this wavelength in a wide wavelength range. Thus any wavelength within the stated bandwidth that is found to produce highest treatment efficiency, can be selected and used. The initial iteration of the new device will have laser emission in the range of 1920-1950 nm. Laser emission is guaranteed to be contained in this wavelength region, but is not fixed down to 1 nm. Other parameters of the initial device from the new design are:

Touchscreen setting;

Power setting for functional laser and for red pointer;

Fixed frequency;

Changeable power and duty cycle (by pulse duration);

Hand piece with diffuser, nice uniform spot;

Battery operation optional;

On/off button on the hand piece, when pressed, the set parameters are executed.

The novel design of the system includes some features that are well known in the industry but offer novelty to the system design when included in the whole design along with new, inventive features.

For example, the use of transmission diffraction grating in stabilization of the wavelength in known in the industry. However, utilizing this method for achieving maximum therapeutic effect observed at certain wavelength for a certain specific medical application is very specific to the new device. The transmission grating/wavelength stabilizing element is also used as an out-coupler mirror for a gain chip when placed close to or adjacent to the AR-coated, anti-reflective coating, front facet of a gain chip. This is novel and specific to the new device. The use of the grating and collimating lens in combination, and using the single integrated optical element at the same time is specific to the new device offering Wavelength stabilizing element, Output coupler mirror for a gain chip when placed close to or adjacent to the output facet and coupling optics for the laser beam.

A further example is precise output power control using a power monitor. This is known in the industry. Measuring a fraction of the light at the exit of the laser module and using this method for precise power control for low-power therapeutic application, where exact dosage is important, is novel and specific to the new device. The ability to adjust the power level over the lifespan of the laser diode, thus keeping calibration of the device for a long time, thus guaranteeing the same therapeutic effects over the lifetime of the system, without re-calibration is specific to the new device. The new device claims no re-calibration of the system during its lifespan even when the fiber replacement is needed or after replacement and this is specific to the new device. Additionally, the use of a collimated beam and diffuser for creating beam size and intensity distribution required for a therapeutic application is known in the industry. However, when the collimated beam is created by an integrated passively—aligned collimator, and a diffuser is also used to minimize the effect of beam collimation quality on the resulting intensity distribution, when used for therapeutic application, is novel and specific to the new device.

The compact advanced hand piece or the handheld one-piece laser system are novel inventions. Using a hand piece with multiple features that improve the accuracy and therapeutic efficacy of the device are known in the art. However, these features become novel when they are combined in the same hand piece together with:

Wavelength stabilized, collimated laser at preferred wavelength per treatment protocol

Dichroic mirror used to combine pointed beam and chosen wavelength beam together, and to deflect a fraction of the beam for power monitoring

Power monitoring (reference signal) using deflected beam, directly in the hand piece using a diffuser to:

Create output beam required for a therapeutic application size and intensity distribution;

Minimize imperfections in the collimated beam coming from a wavelength stabilized laser.

The same features described above may also be used in a one-piece handheld laser.

Traditional hand pieces are used only for delivering the light and beam shaping. However, even after precise factory calibration, the power in the hand piece may shift significantly with time due to:

Inevitable laser chip power degradation with time;

Possible shift of focusing optics with time;

Using replaceable fibers where fiber tip surface quality has variations;

Dirt/contamination accumulated on the tips of the fiber after prolonged use.

The design of the new device will solve the noted problems, such as measurement of the power directly from the output beam, in the hand piece. Fraction of the beam is deflected from the main direction and the reference signal is created by a detector in the hand piece, thus, there is no influence of the above negative factors accumulating with time on the power output.

The new novel features and the combination of features noted in the new device are unique for devices where high power in “not” a requirement, while at the same time spectrum stability is needed, (fixed wavelength, smooth beam shape, and no mode hopping). The novel inventiveness of the new device would “not” be applicable or have functionality in high power devices.

The novel inventions incorporated into the new digital design offer an advancement in the generation and emission of the preferred therapeutic wavelengths described. The new invention offers design which bring novelty to the system when the stabilizing wavelength, which uses transmission diffraction in combination with:

Achieving the maximum therapeutic effect that are observed at a certain wavelength for specific medical applications.

The wavelength stabilizing element has a dual purpose as it is also used as an out-coupler mirror for a gain chip when placed close to or adjacent to the AR-coated “front” facet of a gain chip and not the back facet as in the original design.

The use of the wavelength stabilizing element where the grating and collimating lens are combined together, and this single integrated optical element is used at the same time as; a) Wavelength stabilizing element, b) Output coupler mirror for a gain chip when placed close to or adjacent to the output facet, and c) Coupling optics for the laser beam.

“Not” placing the power monitor behind the chip which is inefficient.

Measuring fraction of the light at the exit of the laser module and using the measurement for “precise power control” for low-power therapeutic applications, where exact dosage is an important requirement.

Using the measurement to adjust the power level over the lifespan of the laser diode, thus keeping calibration of the device for an extended time, therefore guaranteeing the same therapeutic effects over the lifetime of the system, without re-calibration.

Measuring the fraction of the light at the exit end of the output fiber, inside the hand piece thereby no re-calibration of the system during its lifespan even when a fiber replacement is needed and the fiber is replaced.

The collimated beam is created by an integrated passively-aligned collimator, and the diffuser is also used to minimize the effects of beam collimation quality on the resulting intensity distribution when used for therapeutic application.

Combining the following features in the same hand piece, /probe; a) Wavelength stabilized, collimated laser as described herein, b) Dichroic mirror used to combine pointed beam and output beam together and to deflect a fraction of said beam for power monitoring, c) Power monitoring (reference signal) using deflected beam, directly in the hand piece, and d) using a diffuser to create output beam required for a therapeutic application size and intensity distribution and minimizing imperfections in the collimated beam coming from a wavelength stabilized laser.

The same features described above for a three-piece device may be used for a handheld one-piece laser.

With the technology development and extensive studies of the efficacy of the method versus power and wavelength, the possibility exists to replace the edge-emitting diodes by the vertical cavity surface emitting laser (VCSEL) diodes, or even by the light emitting diodes (LED). This allows for further miniaturization of the design opening room for personal home use of the devices.

The New Device will provide major advancements in the field of therapeutic lasers and the overall operation of the low power laser system and will also provide the ability to store patient data and storage of selectable protocols for patient treatment(s) via a digital driver. The new system utilizes a digital driver that provides active control of the output power. If necessary, the driver electronics can be switched from 12-bit to 16- bit to accommodate all power-setting accuracy requirements. The driver sets the current, measures power, and then adjusts the current if the power differs from the set point value. This adjustment uses proprietary algorithms, and the adjustment is done fast and smooth, without current spikes that can harm the laser diode. The driver has memory and can store multiple sets of information such as 1) Laser usage time, 2) Settings for the laser power used in operation, 3) Various treatment protocols and patient history, 4) Data storage for multiple patients, 5) and permits sharing of data over the internet and GPS monitoring of the device. The new device will provide touch screen operation. It has been recently discovered that there is a direct correlation between the Schuman Resonance, the lowest resonant frequency of the Earth's electromagnetic field, which is stated to be 7.83 Hz presently, and improved treatment results from application of the new device. The Schuman resonance is known to alter brainwave and neuro-hormonal responses. (The brain is a very sensitive electromagnetic organ.) Global Coherence Monitoring System (GCMS), 2015. It has been demonstrated that the 7.83 frequency has a strong effect on human brain wave frequencies and thus is likely to increase sensitivity to laser treatment tremendously. The use of the Schuman Resonance frequency is a unique discovery offering optimum treatment outcomes from the new device.

It is well known in the art that the infrared part of the electromagnetic spectrum contains specific areas where water is not transparent to the radiation but absorbs the radiation. There are three main water absorptive wavelengths. 1) Approximately 1450 nm with a 22% water absorption, 2) Approximately 2000 nm where the water absorption is 100%, and 3) Approximately 3000 nm where the water absorption is 10,000 times faster than at 2000 nm. The Field of inventions investigating light and water interactions in biological tissue is a new discipline called Aquaphotonics (Understanding water in biology). The Nd: YAG laser operates at around 1320 nm, the Ho: Yag at around 2000 nm, the Er: YAG at approximate 2940 nm, and the Thulium: YAG at 2020 nm. These lasers, as opposed to the new device, are very high power, thermal, ablative lasers used in the field of dermatology, aesthetic surgery, dentistry, orthopedics and ophthalmology as ablative, tissue destroying lasers. The mid infrared area of the electromagnetic spectrum has a 95-100% water absorption between 1900 nm and 2050 nm peaking at 100% at approximately 2000 nm. The new device can operate outside this bandwidth from approximately 2050 nm thru approximately 3200 nm by changing the laser chip material from InP-based structure used for 1900 nm thru 2050 nm, to a GaSb-based structure, that can extend the wavelength range to about 3200 um to enable the device to work in the same fields as the high power, thermal, ablative lasers, all the while maintaining its athermic (non-thermal) therapeutic purposes using a defocused beam and low power.

The new device design makes it possible to make additional models. At least two compact models, in addition to the table model, powered by mains electric or a portable model powered via batteries. 1) A hand-held probe containing the laser+driver and a separate power case will measure approximately 3″×4″×6″ and will be either battery or power supply operated, and 2) A one-piece unit approximately 3″×1″×5″ with battery operation included in one package. A solution based on an integrated optical element (collimator+selective reflection) has a “disadvantage” for systems where high quality of collimating beam is required, but has “no” effect on the performance of the new design for the specific applications of the device. For high quality of the collimated beam, high placement precision of the collimating optics in respect to the emitting facet of the chip is required. In some instances, the distance from the facet to the optical element should be controlled within the sub-micron range. In this case, active alignment of the optics is required. In the new design, when two optical elements, the lens and the reflective element are combined together, and then as a single piece placed close to the output facet of the chip, there is no room for active alignment. Therefore, due to intrinsic tolerances on the dimensions of the optical elements, submicron range alignment is not achievable. Errors in dimensions will lead to the following imperfections in the collimated beam—it will be either more divergent than ideally collimated or slightly focused. This possible non-ideal collimating beam has “no” disadvantages for the new device and application as it is used together with a diffuser. After the diffuser some variations in the quality of the beam before the diffuser are greatly dumped and the output pattern is the same, for all practical purposes.

The new device's novelty and efficacy are due to a combination of factors working together such as wavelength stabilization, emission method of the wavelength, pulse, and power output. The advancements made to these factors offered by the new device with digital performance and novel methods of wavelength stabilization, emission method, power monitoring and output control, memory and data capabilities, and a more bodily sensitive pulse rate provides for unexpected advantages over existing devices and methods associated therewith.

What is claimed is:

1. A device and method for medical applications, comprising:
a laser having a power adjustment unit for adjusting an output power of the laser in real time;
the laser emitting a beam towards a patient; the beam is partially deflected forming a deflected beam; the deflected beam being used for creating a reference signal for the power adjustment unit;
operating the source of the beam at a predetermined level to activate cells.

2. The device of claim 1, wherein the deflected beam provides information about a chip failure.

3. The device of claim 1, wherein the power adjusting is always provided during the device operation thus ensuring a device self-calibration.

4. The device of claim 1, further comprising a photodiode which is used for the output power measurement.

5. The device of claim 1, wherein a dichroic mirror used to combine a pointed beam and the output beam together and to deflect a fraction of a resulted combined beam forms the deflected beam being used for the laser output power adjusting.

6. The device of claim 1, wherein a diffuser is implemented to create the output beam required for a therapeutic application size and intensity distribution and minimizing imperfections in a laser collimated beam.
